(19) United States
(12) Patent Application Publication
(10) Pub. No.: US 2025/0194993 A1
(43) Pub. Date: Jun. 19, 2025

(54) INFORMATION PROCESSING APPARATUS, INFORMATION PROCESSING METHOD, AND NON-TRANSITORY RECORDING MEDIUM

(71) Applicant: NEC Corporation, Minato-ku, Tokyo (JP)

(72) Inventors: Shizuo SAKAMOTO, Tokyo (JP); Tokio UNNO, Tokyo (JP)

(73) Assignee: NEC Corporation, Minato-ku, Tokyo (JP)

(21) Appl. No.: 18/847,810

(22) PCT Filed: Mar. 25, 2022
(86) PCT No.: PCT/JP2022/014511

§ 371 Date: Sep. 17, 2024

(51) Int. Cl. A61B 5/00; G06T 5/50; G06V 10/74

(52) U.S. Cl. CPC A61B 5/442; G06T 5/50; G06V 10/761; G06T 2207/20221

(57) ABSTRACT

An information processing apparatus includes: an acquisition unit that acquires three-dimensional information on a skin; a detection unit that extracts a pattern from the three-dimensional information and that detects a singular area of the pattern; an extraction unit that extracts a singular area pattern of the singular area; and a generation unit that generates a combined pattern image in which the pattern and the singular area pattern are combined, on the basis of the pattern extracted from the three-dimensional information by the detection unit and the singular area pattern extracted by the extraction unit.

### TECHNICAL FIELD

This disclosure relates to technical fields of an information processing apparatus, an information processing method, and a recording medium.

### BACKGROUND ART

Patent Literature 1 describes a technique/technology in which a transducer which sends and receives an ultrasonic wave moves in a horizontal direction below a window where a fingertip is placed to obtain ultrasonic tomographic images when different surfaces of the fingertip are scanned, and the ultrasonic tomographic images are put together to generate a three-dimensional image including a fingerprint of the fingertip, and then, the three-dimensional image of the fingertip obtained in an identifying operation is compared with a three-dimensional image of the fingertip previously registered in a recording device to decide whether or not an identified person is a specific person. Patent Literature 2 describes a technique/technology in which there are provided: a wrinkle/flaw feature extracting means of extracting a wrinkle and a flaw of a finger from a fingerprint image and outputting information on feature points relating to the wrinkle and flaw; a fingerprint data storage means of storing the information on the feature points relating to the wrinkle and flaw from the wrinkle/flaw feature extracting means as fingerprint data together with information on the feature points of fingerprint ridges; and a collating means of collating the fingerprint based upon the fingerprint data stored in the fingerprint data storage means, the information on the feature points of the fingerprint ridges inputted at collation, and the information on the feature points relating to the wrinkle and flaw, thereby performing precise matching even a fingerprint of low quality such that a surface of the finger is rough or flawed. Patent Literature 3 describes a biological pattern information processing device equipped with: a biological pattern information acquisition unit for acquiring biological pattern information indicating a biological pattern; and a unique region detection unit for detecting, from the biological pattern indicated by the acquired biological pattern information, a unique region including a damaged part.

### CITATION LIST

### Patent Literature

Patent Literature 1: JP2002-175529A
Patent Literature 2: JP2005-352712A
Patent Literature 3: International Publication No. WO2016/159052

### SUMMARY

### Technical Problem

It is an example object of this disclosure to provide an information processing apparatus, an information processing method, and a recording medium that aim to improve the techniques/technologies disclosed in Citation List.

### Solution to Problem

An information processing apparatus according to an example aspect of this disclosure includes: an acquisition unit that acquires three-dimensional information on a skin; a detection unit that extracts a pattern from the three-dimensional information and that detects a singular area of the pattern; an extraction unit that extracts a singular area pattern of the singular area; and a generation unit that generates a combined pattern image in which the pattern and the singular area pattern are combined, on the basis of the pattern extracted from the three-dimensional information by the detection unit and the singular area pattern extracted by the extraction unit.

An information processing method according to an example aspect of this disclosure includes: acquiring three-dimensional information on a skin; extracting a pattern from the three-dimensional information and detecting a singular area of the pattern; extracting a singular area pattern of the singular area; and generating a combined pattern image in which the pattern and the singular area pattern are combined, on the basis of the pattern extracted from the three-dimensional information and the singular area pattern extracted from the singular area.

A recording medium according to an example aspect of this disclosure is a recording medium on which a computer program that allows a computer to execute an information processing method is recorded, the information processing method including: acquiring three-dimensional information on a skin; extracting a pattern from the three-dimensional information and detecting a singular area of the pattern; extracting a singular area pattern of the singular area; and generating a combined pattern image in which the pattern and the singular area pattern are combined, on the basis of the pattern extracted from the three-dimensional information and the singular area pattern extracted from the singular area.

### BRIEF DESCRIPTION OF DRAWINGS

FIG. 1 is a block diagram illustrating a configuration of an information processing apparatus in a first example embodiment.

FIG. 2 is a block diagram illustrating a configuration of an information processing apparatus in a second example embodiment.

FIG. 3 is a flowchart illustrating a flow of an information processing operation performed by the information processing apparatus in the second example embodiment.

FIG. 4 is a conceptual diagram illustrating the information processing operation performed by the information processing apparatus in the second example embodiment.

FIG. 5 is a flowchart illustrating a flow of an information processing operation performed by an information processing apparatus in a third example embodiment.

FIG. 6 is a block diagram illustrating a configuration of an information processing apparatus in a fourth example embodiment.

FIG. 7 is a flowchart illustrating a flow of an information processing operation performed by the information processing apparatus in the fourth example embodiment.

FIG. 8 is a block diagram illustrating a configuration of an information processing apparatus in a fifth example embodiment.

FIG. 9 is a flowchart illustrating a flow of an information processing operation performed by the information processing apparatus in the fifth example embodiment.

FIG. 10 is a block diagram illustrating a configuration of an information processing apparatus in a sixth example embodiment.

FIG. 11 is a flowchart illustrating a flow of an information processing operation performed by the information processing apparatus in the sixth example embodiment.

FIG. 12 is a block diagram illustrating a configuration of an information processing system in a seventh example embodiment.

FIG. 13 is an external view illustrating an appearance of a three-dimensional information generation apparatus in the seventh example embodiment.

### DESCRIPTION OF EXAMPLE EMBODIMENTS

Hereinafter, an information processing apparatus, an information processing method, and a recording medium according to example embodiments will be described with reference to the drawings.

### 1: First Example Embodiment

An information processing apparatus, an information processing method, and a recording medium according to a first example embodiment will be described. The following describes the information processing apparatus, the information processing method, and the recording medium according to the first example embodiment, by using an information processing apparatus 1 to which the information processing apparatus, the information processing method, and the recording medium according to the first example embodiment are applied.

### [1-1: Configuration of Information Processing Apparatus 1]

With reference to FIG. 1, a configuration of the information processing apparatus 1 in the first example embodiment will be described. FIG. 1 is a block diagram illustrating the configuration of the information processing apparatus 1 in the first example embodiment.

As illustrated in FIG. 1, the information processing apparatus 1 includes an acquisition unit 11, a detection unit 12, an extraction unit 13, and a generation unit 14. The acquisition unit 11 acquires three-dimensional information 3DI on a skin. The detection unit 12 extracts a pattern from the three-dimensional information 3DI and detects a singular area of the pattern. The extraction unit 13 extracts a singular area pattern of the singular area. The generation unit 14 generates a combined pattern image in which the pattern and the singular area pattern are combined, on the basis of the pattern extracted from the three-dimensional information 3DI by the detection unit 12 and the singular area pattern extracted from the extraction unit 13.

### [1-2: Technical Effect of Information Processing Apparatus 1]

Since the information processing apparatus 1 in the first example embodiment generates the combined pattern image in which the pattern and the singular area pattern are combined, it is possible to acquire an accurate pattern image with a large amount of information about the pattern, as compared with when the pattern and the singular area pattern are not combined.

### 2: Second Example Embodiment

An information processing apparatus, an information processing method, and a recording medium according to a second example embodiment will be described. The following describes the information processing apparatus, the information processing method, and the recording medium according to the second example embodiment, by using an information processing apparatus 2 to which the information processing apparatus, the information processing method, and the recording medium according to the second example embodiment are applied.

### [2-1: Configuration of Information Processing Apparatus 2]

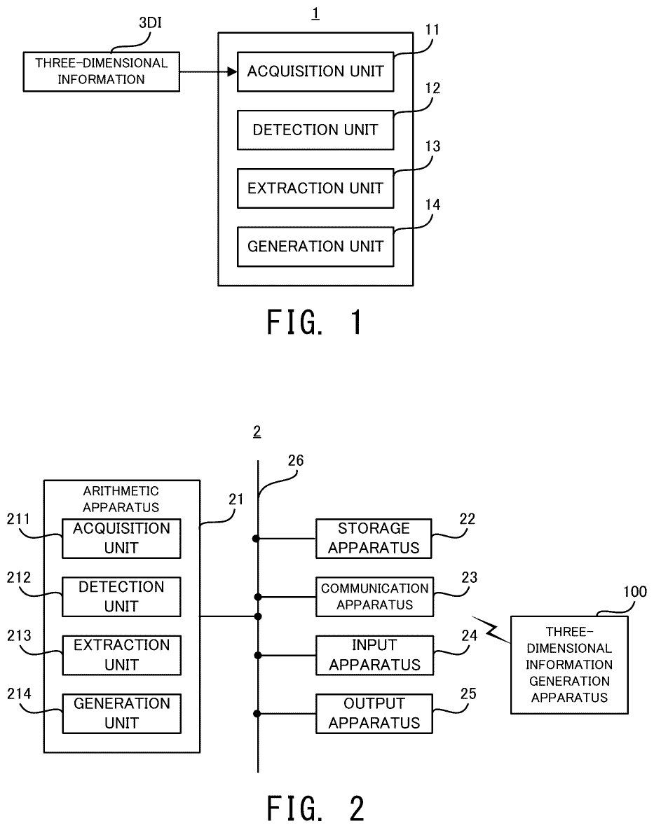

With reference to FIG. 2, a configuration of the information processing apparatus 2 in the second example embodiment will be described. FIG. 2 is a block diagram illustrating the configuration of the information processing apparatus 2 in the second example embodiment.

As illustrated in FIG. 2, the information processing apparatus 2 includes an arithmetic apparatus 21 and a storage apparatus 22. Furthermore, the information processing apparatus 2 may include a communication apparatus 23, an input apparatus 24, and an output apparatus 25. The information processing apparatus 2, however, may not include at least one of the communication apparatus 23, the input apparatus 24, and the output apparatus 25. The arithmetic apparatus 21, the storage apparatus 22, the communication apparatus 23, the input apparatus 24, and the output apparatus 25 may be connected through a data bus 26.

The arithmetic apparatus 21 includes at least one of a CPU (Central Processing Unit), a GPU (Graphics Processing Unit), and a FPGA (Field Programmable Gate Array), for example. The arithmetic apparatus 21 reads a computer program. For example, the arithmetic apparatus 21 may read a computer program stored in the storage apparatus 22. For example, the arithmetic apparatus 21 may read a computer program stored by a computer-readable and non-transitory recording medium, by using a not-illustrated recording medium reading apparatus provided in the information processing apparatus 2 (e.g., the input apparatus 24 described later). The arithmetic apparatus 21 may acquire (i.e., download or read) a computer program from a not-illustrated apparatus disposed outside the information processing apparatus 2, through the communication apparatus 23 (or another communication apparatus). The arithmetic apparatus 21 executes the read computer program. Consequently, a logical functional block for performing an operation to be performed by information processing apparatus 2 is realized or implemented in the arithmetic apparatus 21. That is, the arithmetic apparatus 21 is allowed to function as a control unit for realizing or implementing the logical functional block for performing an operation (in other words, processing) to be performed by the information processing apparatus 2.

FIG. 2 illustrates an example of the logical functional block realized or implemented in the arithmetic apparatus 21 to perform an information processing operation. As illustrated in FIG. 2, an acquisition unit 211 that is a specific example of the “acquision unit”, a detection unit 212 that is a specific example of the “detection unit”, an extraction unit 213 that is a specific example of the “extraction unit”, and a generation unit 214 that is a specific example of the “generation unit” are realized or implemented in the arithmetic apparatus 21. Each operation of the acquisition unit 211, the detection unit 212, the extraction unit 213, and the generation unit 214 will be described later.

The storage apparatus 22 is configured to store desired data. For example, the storage apparatus 22 may temporarily store a computer program to be executed by the arithmetic apparatus 21. The storage apparatus 22 may temporarily store data that are temporarily used by the arithmetic apparatus 21 when the arithmetic apparatus 21 executes the computer program. The storage apparatus 22 may store data that are stored by the information processing apparatus 2 for a long time. The storage apparatus 22 may include at least one of a RAM (Random Access Memory), a ROM (Read Only Memory), a hard disk apparatus, a magneto-optical disk apparatus, a SSD (Solid State Drive), and a disk array apparatus. That is, the storage apparatus 22 may include a non-transitory recording medium.

The communication apparatus 23 is configured to communicate with an apparatus external to the information processing apparatus 2 through a not-illustrated communication network. The communication apparatus 23 may acquire the three-dimensional information 3DI used for the information processing operation, from a three-dimensional information generation apparatus 100 described later, through the communication network.

The input apparatus 24 is an apparatus that receives an input of information to the information processing apparatus 2 from an outside of the information processing apparatus 2. For example, the input apparatus 24 may include an operating apparatus (e.g., at least one of a keyboard, a mouse, and a touch panel) that is operable by an operator of the information processing apparatus 2. For example, the input apparatus 24 may include a reading apparatus that is configured to read information recorded as data on a recording medium that is externally attachable to the information processing apparatus 2.

The output apparatus 25 is an apparatus that outputs information to the outside of the information processing apparatus 2. For example, the output apparatus 25 may output information as an image. That is, the output apparatus 25 may include a display apparatus (a so-called display) that is configured to display an image indicating the information that is desirably outputted. For example, the output apparatus 25 may output information as audio/sound. That is, the output apparatus 25 may include an audio apparatus (a so-called speaker) that is configured to output audio/sound. For example, the output apparatus 25 may output information onto a paper surface. That is, the output apparatus 25 may include a print apparatus (a so-called printer) that is configured to print desired information on the paper surface.

### [2-2: Three-Dimensional Information Generation Apparatus 100]

The three-dimensional information generation apparatus 100 may include, for example, a projection apparatus, an imaging apparatus, and an imaging processing apparatus that processes an image acquired by the imaging apparatus. The projection apparatus may project a first light pattern in which luminance changes in a first period, and a second light pattern in which the luminance changes in a second period that is longer than the first period. The imaging apparatus may acquire an image of a measurement subject on which the first or second light pattern is projected. The imaging processing apparatus may calculate a relative phase value of each part of the measurement subject on the basis of a luminance value of the image of the measurement subject on which the first light pattern is projected, may calculate an absolute phase value of each part of the measurement subject on the basis of the luminance value and the relative phase value of the image of the measurement subject on which the second light pattern is projected, and may calculate three-dimensional coordinates of each part of the measurement subject on the basis of the absolute phase value.

The three-dimensional information generation apparatus 100 may output the three-dimensional coordinates of each part of the measurement subject, as the three-dimensional information 3DI. The above-described configuration of the three-dimensional information generation apparatus 100 is merely an example. For example, the three-dimensional information generation apparatus 100 may be an optical coherence tomography apparatus that generates three-dimensional luminance data on the measurement subject, which are generated by irradiating the measurement subject with a light beam while performing two-dimensional scanning, and by performing optical interference tomography, and may output the three-dimensional luminance data as the three-dimensional information 3DI.

### [2-3: Information Processing Operation Performed by Information Processing Apparatus 2]

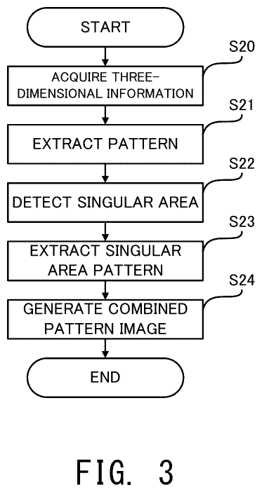

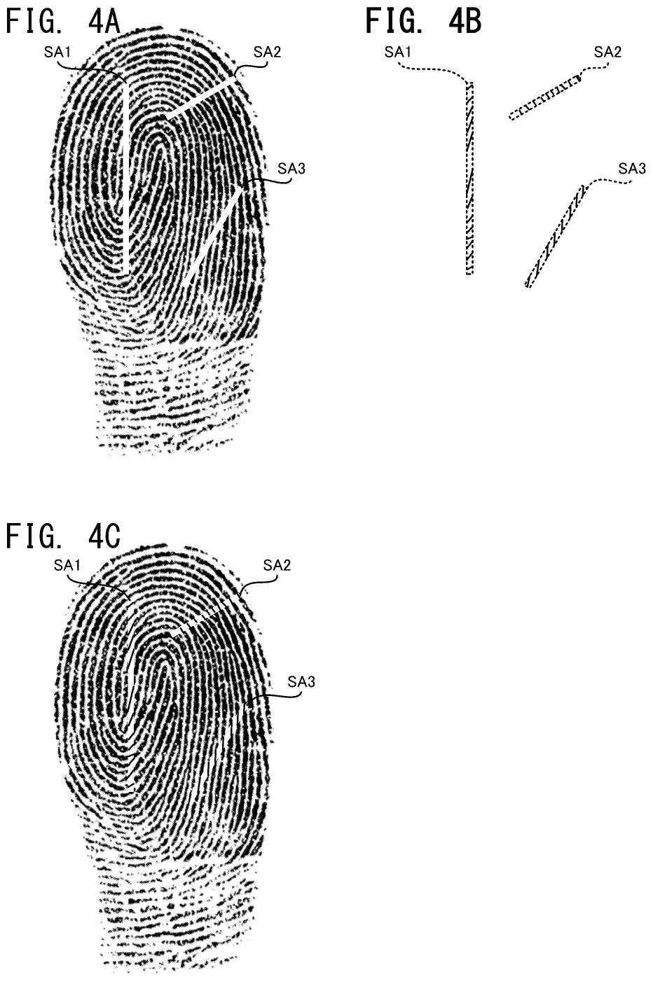

With reference to FIG. 3, a flow of an information processing operation performed by the information processing apparatus 2 in the second example embodiment will be described. FIG. 3 is a flowchart illustrating the flow of the information processing operation performed by the information processing apparatus 2 in the second example embodiment. FIG. 4 is a conceptual diagram illustrating the flow of the information processing operation performed by the information processing apparatus 2 in the second example embodiment.

As illustrated in FIG. 3, the acquisition unit 211 acquires the three-dimensional information 3DI on the skin (step S20). In the second example embodiment, the skin may be a skin of a finger.

The detection unit 212 extracts the pattern from the three-dimensional information 3DI (step S21). In the second example embodiment, the pattern may be a fingerprint. The detection unit 212 may extract a convex part that is in contact with a predetermined surface. The predetermined surface may be a curved surface along a schematic shape of the finger. The convex part may be a ridge. That is, in the step S21, for example, when a seal is stamped, the detection unit 212 may collect information such as information indicating a shape formed on a surface of where the seal is stamped.

The detection unit 212 detects the singular area of the extracted pattern (step S22). The singular area is an area recessed from its surroundings and may include a wrinkle area. The singular area may be an abnormal area of the pattern in which the pattern is discontinuous. An area in which the pattern is not extractable by the detection unit 212, may be a noise area in pattern extraction.

The detection unit 212 may detect an area that is not in contact with the predetermined surface, as the singular area. The singular area may be a notched area in which there is nothing in the image and that is denoted by signs SA1, SA2, and SA3 in FIG. 4(a). For example, the detection unit 212 may detect an area in which the pattern with a predetermined pitch is not extractable, as the singular area. The detection unit 212 may extract an area with an abnormal pattern, from the three-dimensional information 3DI.

The extraction unit 213 extracts the singular area pattern of the singular area (step S23). The extraction unit 213 may estimate an approximate three-dimensional shape of the corresponding singular area on the basis of information about a width of the corresponding singular area and a depth of the most recessed part, and may extract a shape of a convex part that is in contact with the approximate three-dimensional shape. The extraction unit 213 may extract the singular area pattern of each of the singular areas SA1, SA2, and SA3 as illustrated in FIG. 4(b), for example.

The generation unit 214 generates the combined pattern image in which the pattern and the singular area pattern are combined, on the basis of the pattern extracted from the three-dimensional information 3DI by the detection unit 212 and the singular area pattern extracted by the extraction unit 213 (step S24). Based on the pattern extracted from the three-dimensional 3DI by the detection unit 212 and the singular area pattern extracted by the extraction unit 213, the combined pattern image in which the pattern is complemented by the singular area pattern may be generated. The generation unit 214 may generate a combined fingerprint image, for example, as illustrated in FIG. 4(c).

Furthermore, the output apparatus 25 may output the combined pattern image in which the pattern is complemented by the singular area pattern, as illustrated in FIG. 4(c). A singular area part may be displayed in different colors and outputted to be easily visually recognized.

A pattern of epidermis is formed by a pattern of dermis. On the other hand, a recess such as wrinkles is a change in the epidermis. Therefore, even when wrinkles or the like are formed and the recess is generated in the epidermis, the epidermis has the pattern. In other words, a gap between epidermal valleys also has a ridge pattern. In the singular area, the pattern does not appear on a surface at the same height of an area in which no wrinkles are formed, but there is the pattern. In the second example embodiment, the pattern on a surface at a different height is also used to generate the pattern image.

### [2-4: Technical Effect of Information Processing Apparatus 2]

Due to aging, a dry skin condition, or the like, the pattern on the surface of the epidermis may be different from an original pattern. For example, it corresponds to a case where there is an area in which wrinkles or the like are formed on the skin and the epidermis is recessed from the surroundings. This corresponds to a case where a surface condition of the epidermis is different from that of the dermis. In this case, the ridge of the pattern may be broken. This may make it hard to extract an accurate feature point of the pattern. For example, it may lead to a reduction in verification accuracy in verification of the pattern. For example, in fingerprint collection by a two-dimensional scanner, when there are wrinkles on the finger, the ridge is broken by the wrinkles. In addition, in a fingerprint collected by applying ink, a fingerprint acquired by using a live scanner, or the like, it is possible to collect only the pattern in a part that is in contact with a plane surface. That is, since the wrinkle area of the skin is not in contact with the plane surface, the fingerprint in the wrinkle area is not collectable in some cases.

Since the information processing apparatus 2 in the second example embodiment combines and complements the pattern on the surface of the epidermis and the pattern in the area recessed from the surroundings including the wrinkle area, it is possible to acquire an accurate pattern image. That is, the information processing apparatus 2 is capable of reproducing a condition of the dermis.

### 3: Third Example Embodiment

An information processing apparatus, an information processing method, and a recording medium according to a third example embodiment will be described. The following describes the information processing apparatus, the information processing method, and the recording medium according to the third example embodiment, by using an information processing apparatus 3 to which the information processing apparatus, the information processing method, and the recording medium according to the third example embodiment are applied.

The information processing apparatus 3 in the third example embodiment is different from the information processing apparatus 2 in the second example embodiment, in a generation operation performed by the generator 214. Other features of the information processing apparatus 3 may be the same as those of the information processing apparatus 2.

[3-1: Information processing Operation Performed by Information processing Apparatus 3]

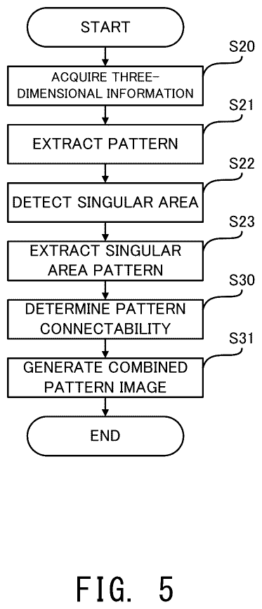

With reference to FIG. 5, a flow of an information processing operation performed by the information processing apparatus 3 in the third example embodiment will be described. FIG. 5 is a flowchart illustrating the flow of the information operation performed by the information processing apparatus 3 in the third example embodiment.

The acquisition unit 211 acquires the three-dimensional information 3DI on the skin (step S20). The detection unit 212 extracts the pattern from the three-dimensional information 3DI (step S21). The detection unit 212 detects the singular area of the pattern (step S22). The extraction unit 213 extracts the singular area pattern of the singular area (step S23).

The generation unit 214 determines whether or not the singular area pattern of each singular area is connectable to a pattern of an area adjacent to the singular area (step S30). The generation unit 214 may determine whether or not the singular area pattern of the singular area is connectable to the pattern of the area adjacent to the singular area, when the pattern and the singular area pattern are combined. When the singular area pattern of the singular area is not connectable to the pattern of the area adjacent to the singular area, the generation unit 214 may correct the shape of a combination part of the corresponding singular area pattern so as to be connectable to the pattern of the adjacent area. For example, the generation unit 214 may correct a line direction of the singular area pattern in the combination part, in accordance with the width and depth of the singular area.

The generation unit 214 generates the combined pattern image in which the pattern and the singular area pattern are combined, on the basis of the singular area pattern that is connectable to the pattern of the area adjacent to the singular area (step S31).

### [3-2: Technical Effect of Information Processing Apparatus 3]

Since it is desirable to combine and complement the broken patterns to acquire the pattern that is the same as or similar to that of the dermis, when the patterns are not connectable and are not useful for complementing broken information, the information processing apparatus 3 in the third example embodiment excludes the singular area pattern from a combination target. When a different pattern from the original pattern is generated, the reliability of the generated pattern image may be reduced. In contrast, the information processing apparatus 3 in the third example embodiment is capable of preventing a reduction in the reliability of the generated pattern image.

### 4: Fourth Example Embodiment

An information processing apparatus, an information processing method, and a recording medium according to a fourth example embodiment will be described. The following describes the information processing apparatus, the information processing method, and the recording medium according to the fourth example embodiment, by using an information processing apparatus 4 to which the information processing apparatus, the information processing method, and the recording medium according to the fourth example embodiment are applied.

### [4-1: Configuration of Information Processing Apparatus 4]

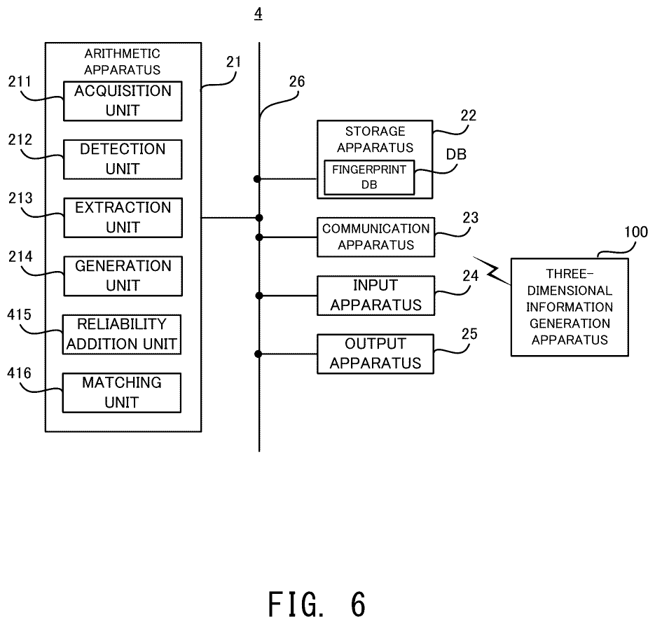

With reference to FIG. 6, a configuration of the information processing apparatus 4 in the fourth example embodiment will be described. FIG. 6 is a block diagram illustrating the configuration of the information processing apparatus 4 in the fourth example embodiment.

As illustrated in FIG. 6, the information processing apparatus 4 in the fourth example embodiment is different from the information processing apparatus 2 in the second example embodiment and the information processing apparatus 3 in the third example embodiment, in that the arithmetic apparatus 21 includes a reliability addition unit 415 and a matching unit 416, and in that the storage apparatus 22 stores a fingerprint database DB in which a registration pattern image is registered in advance. Other features of the information processing apparatus 4 may be the same as those of at least one of the information processing apparatus 2 and the information processing apparatus 3.

### [4-2: Information Processing Operation Performed by Information Processing Apparatus 4]

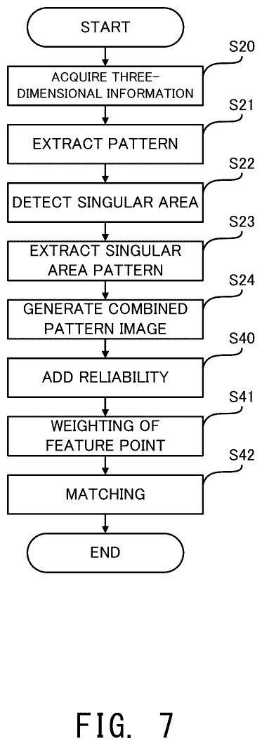

With reference to FIG. 7, a flow of an information processing operation performed by the information processing apparatus 4 in the fourth example embodiment will be described. FIG. 7 is a flowchart illustrating the flow of the information operation performed by the information processing apparatus 4 in the fourth example embodiment.

The acquisition unit 211 acquires the three-dimensional information 3DI on the skin (step S20). The detection unit 212 extracts the pattern from the three-dimensional information 3DI (step S21). The detection unit 212 detects the singular area of the pattern (step S22). The extraction unit 213 extracts the singular area pattern of the singular area (step S23). The generation unit 214 generates the combined pattern image in which the pattern and the singular area pattern are combined, on the basis of the pattern extracted from the three-dimensional information 3DI by the detection unit 212 and the singular area pattern extracted by the extraction unit 213 (step S24).

The reliability addition unit 415 adds higher reliability as a degree of the recess is shallower than that in the surroundings of the singular area (step S40). When the recess is deep in the singular area, the skin tilts significantly, and thus, the reliability of the included pattern may be lower than that in an area without a recess. For example, even successful combination may result in a failure in matching with a registration image. Therefore, the reliability addition unit 415 may add higher reliability as the degree of the recess is shallower than that in the surroundings of the singular area.

The matching unit 416 changes weighting of a feature point included in the combined fingerprint image in accordance with the reliability (step S41). The feature point may include an “edge point” that is a point at which the pattern is broken, and a “branch point” that is a point at which the pattern is branched.

The matching unit 416 matches the combined fingerprint image with the registration pattern image registered in advance (step S42). The generation unit 214 may further generate a ridge image in which the ridge is extracted from the combined pattern image. The generation unit 214 may further generate a two-dimensional image in which only the ridge is left from the combined pattern image. The matching unit 416 may match the ridge image with the registration pattern image registered in advance.

In addition, the output apparatus 25 may output information about the width and depth of the singular area, and information related to a singular point of the pattern included in the singular area.

### [4-3: Technical Effect of Information Processing Apparatus 4]

In many cases, as deformation of a surface shape is smaller, it is easier to reproduce a state when the surface shape is not deformed. On the contrary, in many cases, as the deformation of the surface shape is larger, it is harder to reproduce the state when the surface shape is not deformed. That is, in many cases, as the deformation of the surface shape is smaller, the reproduced pattern may be more reliable, and on the contrary, as the deformation of the surface shape is larger, the reproduced pattern may be unreliable. The information processing apparatus 4 in the fourth example embodiment is capable of effectively utilizing the generated pattern image, by adding the reliability to the reproduced pattern. Especially in the verification, information that allows determination of whether or not a verification result is reliable, is useful. Furthermore, since the information processing apparatus 4 further generates a ridge image in which the ridge is extracted from the combined pattern image, it is possible to obtain an image suitable for the verification.

### 5: Fifth Example Embodiment

An information processing apparatus, an information processing method, and a recording medium according to a fifth example embodiment will be described. The following describes the information processing apparatus, the information processing method, and the recording medium according to the fifth example embodiment, by using an information processing apparatus 5 to which the information processing apparatus, the information processing method, and the recording medium according to the fifth example embodiment are applied.

### [5-1: Configuration of Information Processing Apparatus 5]

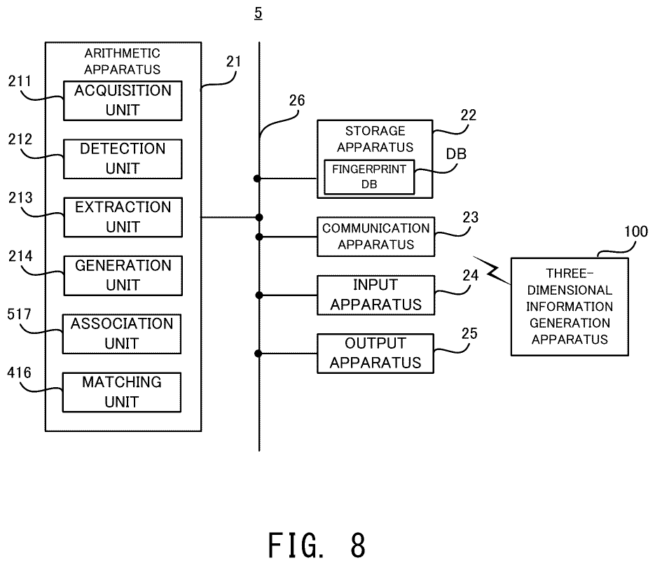

With reference to FIG. 8, a configuration of the information processing apparatus 5 in the fifth example embodiment will be described. FIG. 8 is a block diagram illustrating the configuration of the information processing apparatus 5 in the fifth example embodiment.

As illustrated in FIG. 8, the information processing apparatus 5 in the fifth example embodiment is different from the information processing apparatus 2 in the second example embodiment to the information processing apparatus 4 in the fourth example embodiment, in that the arithmetic apparatus 21 includes the matching unit 416 and an association unit 517, and in that the storage apparatus 22 stores the fingerprint database DB in which the registration pattern image is registered in advance. Other features of the information processing apparatus 5 may be the same as those of at least one of the information processing apparatus 2 to the information processing apparatus 4.

### [5-2: Information Processing Operation Performed by Information Processing Apparatus 5]

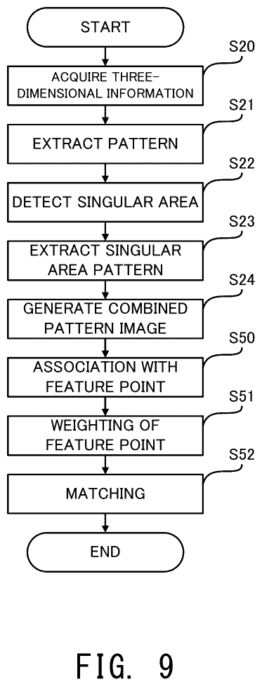

With reference to FIG. 9, a flow of an information processing operation performed by the information processing apparatus 5 in the fifth example embodiment will be described. FIG. 9 is a flowchart illustrating the flow of the information processing operation performed by the information processing apparatus 5 in the fifth example embodiment.

The acquisition unit 211 acquires the three-dimensional information 3DI on the skin (step S20). The detection unit 212 extracts the pattern from the three-dimensional information 3DI (step S21). The detection unit 212 detects the singular area of the pattern (step S22). The extraction unit 213 extracts the singular area pattern of the singular area (step S23). The generation unit 214 generates the combined pattern image in which the pattern and the singular area pattern are combined, on the basis of the pattern extracted from the three-dimensional information 3DI by the detection unit 212 and the singular area pattern extracted by the extraction unit 213 (step S24).

When the feature point included in the combined fingerprint image is in an area based on the singular area pattern, the association unit 517 associates singular area information indicating that the feature point is based on the singular area pattern, with the corresponding feature point (step S50).

The association unit 517 reduces the weighting of the feature point with which the singular area information is associated (step S51).

The association unit 517 matches the combined fingerprint image with the registration pattern image registered in advance (step S52). The generation unit 214 may further generate the ridge image in which the ridge is extracted from the combined pattern image. The association unit 517 may match the ridge image with the registration pattern image registered in advance.

### [5-3: Technical Effect of Information Processing Apparatus 5]

The feature point of the pattern image may be a position where a feature of the pattern image is allowed to be captured well. For example, it may be a position used to compare the pattern images when the pattern image are collated/verified. Therefore, in many cases, a more reliable location is preferably used as a position where the feature of the printed image is allowed to be captured well. In many cases, a position extracted as the feature point is derived from which area, i.e., whether the position is derived from an area other than the singular area, or from the singular area, is preferably distinguished. The information processing apparatus 5 in the fifth example embodiment is capable of distinguishing whether the position is derived from the area origin other than the singular area, or from the singular area. Since the information processing apparatus 5 associates the feature point with the singular area information indicating that the feature point is based on the singular area pattern, it is possible to determine whether or not the corresponding position is used in processing as the feature point, in accordance with the application. Especially in the verification of the pattern images, information that allows determination of whether or not to use the corresponding position for the verification, is useful.

### 6: Sixth Example Embodiment

An information processing apparatus, an information processing method, and a recording medium according to a sixth example embodiment will be described. The following describes the information processing apparatus, the information processing method, and the recording medium according to the sixth example embodiment, by using an information processing apparatus 6 to which the information processing apparatus, the information processing method, and the recording medium according to the sixth example embodiment are applied.

### [6-1: Configuration of Information Processing Apparatus 6]

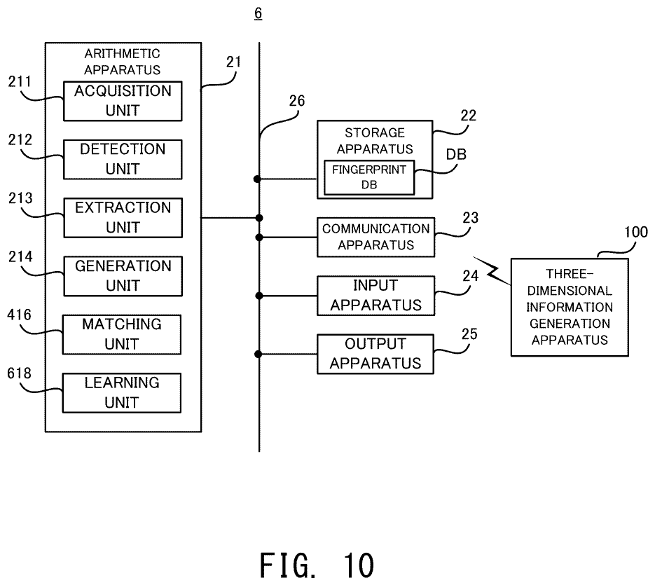

With reference to FIG. 10, a configuration of the information processing apparatus 6 in the sixth example embodiment will be described. FIG. 10 is a block diagram illustrating the configuration of the information processing apparatus 6 in the sixth example embodiment.

As illustrated in FIG. 10, the information processing apparatus 6 in the sixth example embodiment is different from the information processing apparatus 2 in the second example embodiment to the information processing apparatus 5 in the fifth example embodiment, in that the arithmetic apparatus 21 includes the matching unit 416 and a learning unit 618, and in that the storage apparatus 22 stores the fingerprint database DB in which the registration print image is registered in advance. Other features of the information processing apparatus 6 may be the same as those of at least one of the information processing apparatus 2 to the information processing apparatus 5.

### [6-2: Information Processing Operation Performed by Information Processing Apparatus 6]

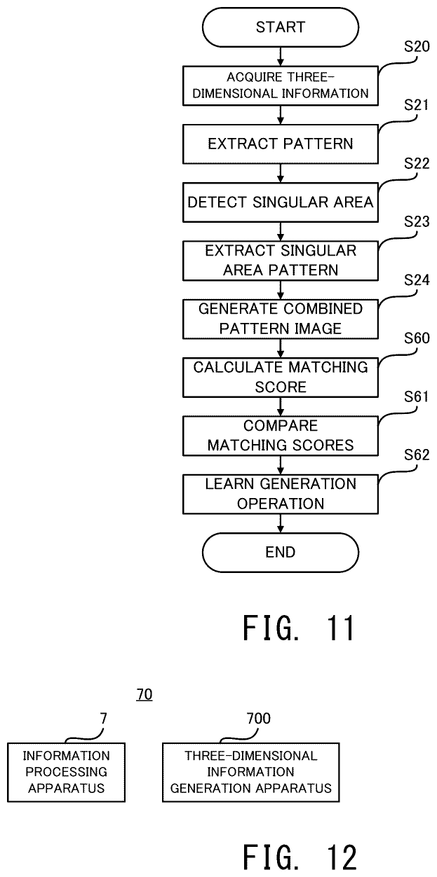

With reference to FIG. 11, a flow of an information processing operation performed by the information processing apparatus 6 in the sixth example embodiment will be described. FIG. 11 is a flowchart illustrating the flow of the information processing operation performed by the information processing apparatus 6 in the sixth example embodiment.

### Acquisition Unit 211 Acquires a Three-Dimensional Information 3DI on the Skin (Step S20).

The acquisition unit 211 acquires the three-dimensional information 3DI on the skin (step S20). The detection unit 212 extracts the pattern from the three-dimensional information 3DI (step S21). The detection unit 212 detects the singular area of the pattern (step S22). The extraction unit 213 extracts the singular area pattern of the singular area (step S23). The generation unit 214 generates the combined pattern image in which the pattern and the singular area pattern are combined, on the basis of the pattern extracted from the three-dimensional information 3DI by the detection unit 212 and the singular area pattern extracted by the extraction unit 213 (step S24).

The matching unit 416 calculates a combined matching score between the combined pattern image and the registration fingerprint image registered in advance, and an uncombined matching score between the pattern image based on the pattern extracted from the three-dimensional information 3DI and the registration fingerprint image registered in advance (step S60).

The learning unit 618 compares the combined matching score with the uncombined matching score (step S61).

When a difference between the combined matching score and the uncombined matching score is smaller than a predetermined value, the learning unit 618 allows the generation section 214 to learn a method of generating the combined pattern image so as to omit a combination between the pattern and the singular area pattern (step S62).

That is, the generation unit 214 may learn the method of generating the combined pattern image so as to combine the pattern and the singular area pattern, in a case where it is possible to obtain an advantageous effect by combining the pattern and the singular area pattern, such as improving the accuracy of the pattern image.

In addition, the learning unit 618 may build an image generation model that allows the combination between the pattern and the singular area pattern, when it is possible to obtain an advantageous effect. The generation unit 214 may generate the combined pattern image in which the pattern and the singular area pattern are combined by using the image generation model. By using the learned image generation model, the generation unit 214 is capable of reducing a processing load of generation of the combined pattern image, even when the information processing apparatus acquires three-dimensional information on an unknown skin.

A parameter that defines the operation of the image generation model may be stored in the storage apparatus 22. The parameter that defines the operation of image generation model may be a parameter updated by a learning operation, and may be a weight and bias of a neural network, for example.

### [6-3: Technical Effect of Information Processing Apparatus 6]

The information processing apparatus 6 in the sixth example embodiment is capable of reducing the processing load by omitting processing, when it is not possible to obtain an advantageous effect even if the pattern and the singular area pattern are combined. The information processing apparatus 6 is capable of omitting the processing when the complement is not required, and is thus capable of performing an efficient operation of generating the pattern image.

### 7: Seventh Example Embodiment

An information processing apparatus, an information processing method, and a recording medium according to a seventh example embodiment will be described. The following describes the information processing apparatus, the information processing method, and the recording medium according to the seventh example embodiment, by using an information processing system 70 to which the information processing apparatus, the information processing method, and the recording medium according to the seventh example embodiment are applied.

### [7-1: Configuration of Information Processing System 70]

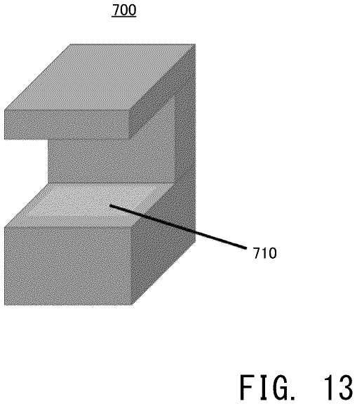

With reference to FIG. 12, a configuration of the information processing system 70 in the seventh example embodiment will be described. FIG. 12 is a block diagram illustrating the configuration of the information processing system 70 in the seventh example embodiment. As illustrated in FIG. 12, the information processing system 70 in the seventh example embodiment may include an information processing apparatus 7 and a three-dimensional information generation apparatus 700.

The information processing apparatus 7 in the seventh example embodiment may be the same as at least one of the information processing apparatus 2 in the second example embodiment to the information processing apparatus 6 in the sixth example embodiment. The information processing apparatus 7 and the three-dimensional information generation apparatus 700 may transmit/receive information through a communication network. Alternatively, the information processing apparatus 7 and the three-dimensional information generation apparatus 700 may be integrally configured. For example, the information processing apparatus 7 may include the three-dimensional information generation apparatus 700. Alternatively, the three-dimensional information generation apparatus 700 may include the information processing apparatus 7.

### [7-2: Three-Dimensional Information Generation Apparatus 700]

With reference to FIG. 13, the three-dimensional information generation apparatus 700 will be described. FIG. 13 is an external view illustrating an appearance of the three-dimensional information generation apparatus 700. The three-dimensional information generation apparatus 700 may include a scanner unit 710. As illustrated in FIG. 13, the three-dimensional information generation apparatus 700 may be configured to have a finger of a hand held over the scanner unit 710, with a palm directed downward.

The scanner unit 710 may be a contactless fingerprint scanner. The scanner unit 710 may capture a three-dimensional fingerprint image as the three-dimensional 3DI on the skin.

The three-dimensional information generation apparatus 700 may capture a plurality of three-dimensional fingerprint images and generate a composite image in which the three-dimensional fingerprint images are combined. The three-dimensional information generation apparatus 700 may output the generated composite image as the three-dimensional information 3DI on the skin.

### [8: Supplementary Notes]

With respect to the example embodiment described above, the following Supplementary Notes are further disclosed.

### [Supplementary Note 1]

An information processing apparatus including:
an acquisition unit that acquires three-dimensional information on a skin;
a detection unit that extracts a pattern from the three-dimensional information and that detects a singular area of the pattern;
an extraction unit that extracts a singular area pattern of the singular area; and
a generation unit that generates a combined pattern image in which the pattern and the singular area pattern are combined, on the basis of the pattern extracted from the three-dimensional information by the detection unit and the singular area pattern extracted by the extraction unit.

### [Supplementary Note 2]

The information processing apparatus according to Supplementary Note 1, wherein the singular area is an area recessed from its surroundings and includes a wrinkle area.

### [Supplementary Note 3]

The information processing apparatus according to Supplementary Note 1 or 2, wherein the generation unit generates the combined pattern image in which the pattern and the singular area pattern are combined, on the basis of a singular area pattern that is connectable to a pattern of an area adjacent to the singular area, out of the singular area pattern.

### [Supplementary Note 4]

The information processing apparatus according to any one of Supplementary Notes 1 to 3, wherein the generation unit further generates a ridge image in which a ridge is extracted from the combined pattern image.

### [Supplementary Note 5]

The information processing apparatus according to any one of Supplementary Notes 1 to 4, further including a reliability addition unit that adds higher reliability as a degree of a recess is shallower than that in surroundings of the singular area.

### [Supplementary Note 6]

The information processing apparatus according to any one of Supplementary Notes 1 to 5, further including an association unit that associates singular area information indicating that a feature point is based on the singular area pattern, with the corresponding feature point, when the feature point included in the combined pattern image is in an area based on the singular area pattern.

### [Supplementary Note 7]

The information processing apparatus according to Supplementary Note 5, further including a matching unit that matches the combined pattern image with a registration pattern image registered in advance, wherein the matching unit changes weighting of a feature point included in the combined pattern image in accordance with the reliability.

### [Supplementary Note 8]

The information processing apparatus according to Supplementary Note claim 6, further including a matching unit that matches the combined pattern image with a registration pattern image registered in advance, wherein the matching unit reduces weighting of the feature point with which the singular area data is associated.

### [Supplementary Note 9]

The information processing apparatus according to any one of Supplementary Notes 1 to 8, further including a learning unit that allows the generation unit to learn a method of generating the combined method image,
such that a combined matching score between the combined pattern image and a registration pattern image registered in advance, and an uncombined matching score between a pattern image based on the pattern extracted from the three-dimensional information and a registration pattern image registered in advance, are calculated, and
so as to omit a combination between the pattern and the singular area pattern, when a difference between the combined matching score and the uncombined matching score is smaller than a predetermined value.

### [Supplementary Note 10]

An information processing method including:
acquiring three-dimensional information on a skin;
extracting a pattern from the three-dimensional information and detecting a singular area of the pattern;
extracting a singular area pattern of the singular area; and
generating a combined pattern image in which the pattern and the singular area pattern are combined, on the basis of the pattern extracted from the three-dimensional information and the singular area pattern extracted from the singular area.

### [Supplementary Note 11]

A recording medium on which a computer program that allows a computer to execute an information processing method is recorded, the information processing method including:
acquiring three-dimensional information on a skin;
extracting a pattern from the three-dimensional information and detecting a singular area of the pattern;
extracting a singular area pattern of the singular area; and
generating a combined pattern image in which the pattern and the singular area pattern are combined, on the basis of the pattern extracted from the three-dimensional information and the singular area pattern extracted from the singular area.

At least a part of the constituent components of each of the example embodiments described above can be combined with at least another part of the constituent components of each of the example embodiments described above, as appropriate. A part of the constituent components of each of the example embodiments described above may not be used. Furthermore, to the extent permitted by law, all the references (e.g., publications) cited in this disclosure are incorporated by reference as a part of the description of this disclosure.

This disclosure is not limited to the examples described above and is allowed to be changed, if desired, without departing from the essence or spirit of this disclosure which can be read from the claims and the entire identification. An information processing apparatus, an information processing method, and a recording medium with such changes are also intended to be within the technical scope of this disclosure.

### DESCRIPTION OF REFERENCE CODES

1, 2, 3, 4, 5, 6, 7 Information processing apparatus
11, 211 Acquisition unit
12, 212 Detection unit
13, 213 Extraction unit
14, 214 Generation unit
415 Reliability addition unit
416 Matching unit
517 Association unit
618 Learning unit
100, 700 Three-dimensional information generation apparatus
710 Scanner unit
70 Information processing system

What is claimed is:

1. An information processing apparatus comprising:
at least one memory that is configured to store instructions; and
at least one processor that is configured to execute the instructions to:
acquire three-dimensional information on a skin;
extract a pattern from the three-dimensional information and detect a singular area of the pattern;
extract a singular area pattern of the singular area; and
generate a combined pattern image in which the pattern and the singular area pattern are combined, on the basis of the pattern extracted from the three-dimensional information and the singular area pattern extracted.

2. The information processing apparatus according to claim 1, wherein the singular area is an area recessed from its surroundings and includes a wrinkle area.

3. The information processing apparatus according to claim 1, wherein the at least one processor is configured to execute the instructions to generate the combined pattern image in which the pattern and the singular area pattern are combined, on the basis of a singular area pattern that is connectable to a pattern of an area adjacent to the singular area, out of the singular area pattern.

4. The information processing apparatus according to claim 1, wherein the at least one processor is configured to execute the instructions to further generate a ridge image in which a ridge is extracted from the combined pattern image.

5. The information processing apparatus according to claim 1, wherein the at least one processor is configured to execute the instructions to add higher reliability as a degree of a recess is shallower than that in surroundings of the singular area.

6. The information processing apparatus according to claim 1, wherein the at least one processor is configured to execute the instructions to associate singular area information indicating that a feature point is based on the singular area pattern, with the corresponding feature point, when the feature point included in the combined pattern image is in an area based on the singular area pattern.

7. The information processing apparatus according to claim 5, wherein the at least one processor is configured to execute the instructions to:
match the combined pattern image with a registration pattern image registered in advance; and
change weighting of a feature point included in the combined pattern image in accordance with the reliability.

8. The information processing apparatus according to claim 6, wherein the at least one processor is configured to execute the instructions to:
match the combined pattern image with a registration pattern image registered in advance; and
reduce weighting of the feature point with which the singular area data is associated.

9. The information processing apparatus according to claim 1, wherein the at least one processor is configured to execute the instructions to allow a method of generating the combined method image to be learned,
such that a combined matching score between the combined pattern image and a registration pattern image registered in advance, and an uncombined matching score between a pattern image based on the pattern extracted from the three-dimensional information and a registration pattern image registered in advance, are calculated, and
so as to omit a combination between the pattern and the singular area pattern, when a difference between the combined matching score and the uncombined matching score is smaller than a predetermined value.

10. An information processing method comprising:
acquiring three-dimensional information on a skin;
extracting a pattern from the three-dimensional information and detecting a singular area of the pattern;
extracting a singular area pattern of the singular area; and
generating a combined pattern image in which the pattern and the singular area pattern are combined, on the basis of the pattern extracted from the three-dimensional information and the singular area pattern extracted from the singular area.

11. A non-transitory recording medium on which a computer program that allows a computer to execute an information processing method is recorded, the information processing method including:
acquiring three-dimensional information on a skin;
extracting a pattern from the three-dimensional information and detecting a singular area of the pattern;
extracting a singular area pattern of the singular area; and
generating a combined pattern image in which the pattern and the singular area pattern are combined, on the basis of the pattern extracted from the three-dimensional information and the singular area pattern extracted from the singular area.
